(19) United States
(12) Patent Application Publication
(10) Pub. No.: US 2025/0136700 A1
(43) Pub. Date: May 1, 2025

(54) BISPECIFIC ANTIBODY FOR CANCER IMMUNOTHERAPY

(71) Applicant: H. LEE MOFFITT CANCER CENTER AND RESEARCH INSTITUTE, INC., Tampa, FL (US)

(72) Inventor: Marco L. Davila, Tampa, FL (US)

(21) Appl. No.: 18/823,163

(22) Filed: Sep. 3, 2024

Related U.S. Application Data
(63) Continuation of application No. 16/487,372, filed on Aug. 20, 2019, now Pat. No. 12,077,590.
(60) Provisional application No. 62/462,057, filed on Feb. 22, 2017.

(51) Int. Cl. C07K 16/28

(52) U.S. Cl. CPC C07K 16/283; C07K 2317/622

(57) ABSTRACT

Disclosed are compositions and methods for gene transfer in immune effector cells, e.g. to create chimeric antigen receptors (CARs). In particular, polypeptides are disclosed that comprising an antigen-binding region and a heparin binding domain, wherein the antigen-binding region is capable of specifically binding to an immune cell antigen located on an immune effector cell, and wherein the heparin binding domain is capable of binding a viral vector.

### CROSS-REFERENCE TO RELATED APPLICATIONS

This application claims benefit of U.S. Provisional Application No. 62/462,057, filed Feb. 22, 2017, incorporated herein by reference in its entirety.

### BACKGROUND

Adoptive T cell immunotherapy offers a promising strategy for specifically targeting and eliminating cancerous cells. T cells can be engineered ex vivo to express chimeric antigen receptors specific for tumor antigens (CAR T cells). The expansion and function of adoptively transferred CAR T cells can be potentiated by the lymphodepletive and tumoricidal effects of standard of care chemotherapy and radiotherapy.

Production of CAR T cells currently involves isolation and activation of T cells from peripheral blood mononuclear cell (PBMC) with anti-CD3/anti-CD28 paramagnetic beads. The T cells are added to culture containers pre-treated with RetroNectin® and incubated with viral particles for gene transfer. The cells, beads and virus are incubated and the gene-modified cells are expanded. The beads and RetroNectin® are expensive and are limited in supply. Furthermore, the paramagnetic beads are also limited in their ability to be conjugated with more antibodies to activate and/or co-stimulate immune cells due to their small size.

### SUMMARY

Disclosed is a less costly, renewable, modifiable, and efficacious alternative to coated beads and RetroNectin® for gene transfer. Disclosed are fusion polypeptides comprising 1) variable domains of antibodies that specifically bind an immune cell antigen and 2) a heparin binding domain (HBD). Antibodies binding immune cell antigens, such as CD3, CD28, or CD137, bind and activate expanding T cells ex vivo, while the HBD binds the viral vector, thereby bringing the T cells into close proximity with viral particles for effective gene transfer.

In some embodiments, the fusion polypeptide has the following formula:

V_{L}I-V_{H}I-HBD-Fc,

V_{H}I-V_{L}I-HBD-Fc,

HBD-V_{L}I-V_{H}I-Fc,

HBD-V_{H}I-V_{L}I-Fc,

Fc-V_{L}I-V_{H}I-HBD,

Fc-V_{H}I-V_{L}I-HBD,

Fc-HBD-V_{L}I-V_{H}I, or

Fc-HBD-V_{H}I-V_{L}I,
wherein “V_{L}I” is a light chain variable domain specific for an immune cell antigen;
wherein “V_{H}I” is a heavy chain variable domain specific for the immune cell antigen;
wherein “HBD” is a heparin binding domain;
wherein “-” consists of a peptide linker or a peptide bond;
wherein “-” consists of a peptide linker or a peptide bond; and
wherein “Fc” consist of an optional Fc domain of an immunoglobulin.

The Fc (“fragment crystalline”) portion of an antibody molecule includes the CH2 and CH3 domains of the heavy chain and a portion of the hinge region. It was originally defined by digestion of an IgG molecule with papain. Fc is responsible for two of the highly desirable properties of an IgG: recruitment of effector function and a long serum half-life. In some embodiments, the polypeptide sequence of an Fc domain is substantially similar to an Fc polypeptide sequence of: an IgG1 Fc region, an IgG2 Fc region, an IgG3 Fc region, an IgG4 Fc region, an IgM Fc region, an IgA Fc region, an IgD Fc region, or an IgE Fc region. In some embodiments, the Fc domain refers to the last two constant region immunoglobulin domains of IgA, IgD, and IgG, and the last three constant region immunoglobulin domains of IgE and IgM. In some embodiments, the disclosed fusion polypeptide can comprise one to four domains selected from the group consisting of CH1, CH2, CH3 and CH4 of IgG1, IgG2, IgG3 or IgG4, and may include a hinge region. The CH1, CH2, and/or CH3 domains may be derived from humans or other animals including cows, goats, swine, mice, rabbits, hamsters, rats and guinea pigs, and preferably humans, or synthetic, or a combination thereof. In addition, the Fc portion may be derived from IgG, IgA, IgD, IgE and IgM, or can be made by combinations thereof or hybrids thereof. Sequences for Fc domains are known in the art and publicly available.

The immune cell antigen can be a cell surface molecule that is expressed on human NK cells, T cells, monocytes, macrophages or granulocytes. For example, the cell surface molecule can be antigen CD2, CD3, CD16, CD64, CD89, CD137 (4-1BB), NKp30, NKp44, NKp46, NKp80 (KLR-F1), NKG2C or NKG2D.

In some embodiments, the anti-CD3 scFv comprises the amino acid sequence:

(SEQ ID NO: 1)
MASPLTRFLSLNLLLLGESIILGSGEAQVQLQESGAELARPGASVKMSCK
ASGYTFTRYTMHWVKQRPGQGLEWIGYINPSRGYTNYNQKFKDKATLTTD
KSSSTAYMQLSSLTSEDSAVYYCARYYDDHYCLDYWGQGTTVTVSSGGGG
SGGGGSGGGGSDIQLTQSPAIMSASPGEKVTMTCSASSSVSYMNWYQQKS
GTSPKRWIYDTSKLASGVPAHFRGSGSGTSYSLTISGMEAEDAATYYCQQ
WSSNPFTFGSGTKLEIKR.

In some embodiments, the anti-CD28 scFv comprises the amino acid sequence:

(SEQ ID NO: 2)
MASPLTRFLSLNLLLLGESIILGSGEAQVQLQQSGTELVKPASSVKISCK
ASGYTSTSNYMHWIRQQPGNGLEWIGRIYPGNGNTKYNQKFDGKATPTAD
KSSSTAYMQLSRLTFEDSAVYFCASAPLDYGGHIMDAWGQGTTVTVSSGG
GGSGGGGSGGGGSDIQLTQSPAFLSASLGETVSIECLGSEDIYGYLAWYQ
QKPGKSPQLLIYVANRLQDGVPSRFSGSGSGTQYSLKISGMQPEDEGDYY
CLQGSKFPLTFGSGTKLEIKR.

In some embodiments, the HBD comprises the amino acid sequence:

(SEQ ID NO: 3)
AIPAPTDLKFTQVTPTSLSAQWTPPNVQLTGYRVRVTPKEKTGPMKEINL
APDSSSVVVSGLMVATKYEVSVYALKDTLTSRPAQGVVTTLENVSPPRRA
RVTDATETTITISWRTKTETITGFQVDAVPANGQTPIQRTIKPDVRSYTI
TGLQPGTDYKIYLYTLNDNARSSPVVIDASTAIDAPSNLRFLATTPNSLL
VSWQPPRARITGYIIKYEKPGSPPREVVPRPRPGVTEATITGLEPGTEYT
IYVIALKNNQKSEPLIGRKKT.

Also disclosed is an isolated nucleic acid encoding the disclosed fusion polypeptide, as well as nucleic acid vectors containing this isolated nucleic acid operably linked to an expression control sequence. Also disclosed are cells transfected with these vectors and the use of these cells to produce the disclosed fusion polypeptides.

An antigen binding molecule can be formed from dimerization of heavy and light chains. In these embodiments, the V_{L}I dimerizes with V_{H}I to form an antigen binding site for an immune cell antigen (e.g., CD3).

Also disclosed is a fusion polypeptide comprising an antigen-binding region capable of recruiting the activity of a human immune effector cell by specifically binding to an immune cell antigen located on the human immune effector cell; and a heparin binding domain.

The antigen-binding region can be derived from natural antibodies, such as monoclonal antibodies. In some cases, the antibody is human. In some cases, the bispecific antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Currently, the most widely used technique for antibody human adaptation is known as “CDR grafting.” The scientific basis of this technology is that the binding specificity of an antibody resides primarily within the three hypervariable loops known as the complementarity determining regions (CDRs) of its light and heavy chain variable regions (V-regions), whereas the more conserved framework regions (framework, FW; framework region, FR) provide structure support function. By grafting the CDRs to an appropriately selected FW, some or all of the antibody-binding activity can be transferred to the resulting recombinant antibody.

CDR grafting is the selection of a most appropriate human antibody acceptor for the graft. Various strategies have been developed to select human antibody acceptors with the highest similarities to the amino acid sequences of donor CDRs or donor FW, or to the donor structures. All these “best fit” strategies, while appearing very rational, are in fact based on one assumption, i.e., a resulting recombinant antibody that is most similar (in amino acid sequence or in structure) to the original antibody will best preserve the original antigen binding activity.

Not all amino acids in the CDRs are involved in antigen binding. Thus, it has been proposed that the grafting of only those residues that are critical in antigen-antibody interaction—the so-called specificity determining residues grafting (SDR-grafting)-will further increase the content of human antibody sequences in the resulting recombinant antibody. The application of this strategy requires information on the antibody structure as well as antibody-antigen contact residues, which are quite often unavailable. Even when such information is available, there is no systematic method to reliably identify the SDRs, and SDR-grafting remains so far mostly at the basic research level.

Recently, a strategy called “human framework shuffling” has been developed. This technique works by ligating DNA fragments encoding CDRs to DNA fragments encoding human FR1, FR2, FR3, and FR4, thus generating a library of all combinations between donor CDRs and human FRs. Methods for making human-adapted antibodies based on molecular structures, modeling and sequences for human engineering of antibody molecules are disclosed in U.S. Pat. No. 8,748,356, which is incorporated by reference for these methods.

The effector cell recruited by the bispecific antibody is one capable of exerting a cytotoxic or an apoptotic effect on a target cell. In some embodiments, the human effector cell can in some embodiments be a member of the human lymphoid lineage or myeloid lineage. As an example, for lymphoid cells, the immune cell antigen can be selected from the group consisting of human CD3 antigen, human CD16 antigen, human NKG2D antigen, human CD2 antigen, human CD28 antigen, and human CD25 antigen. Similarly, for myeloid cells, the immune cell antigen can be human CD64 antigen or human CD89 antigen.

Also disclosed is a pharmaceutical composition comprising a molecule disclosed herein in a pharmaceutically acceptable carrier.

Also disclosed herein are methods for treating cancer in a subject comprising administering to the subject a therapeutically effective amount of a disclosed pharmaceutical composition.

Also disclosed is a kit comprising a bispecific antibody disclosed herein.

Also disclosed is an expression vector comprising an isolated nucleic acid encoding a bispecific antibody disclosed herein operably linked to an expression control sequence. Also disclosed is a cell comprising the disclosed expression vector. The cell can be a primary cell, transformed cell, cell line, or the like. In some cases, the cell is a mammalian cell line. In some cases, the cell is a non-mammalian cell line. For example, the cell can be a bacteria or insect cell line.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates the prior art method for CAR T cell production with beads and retronectin.

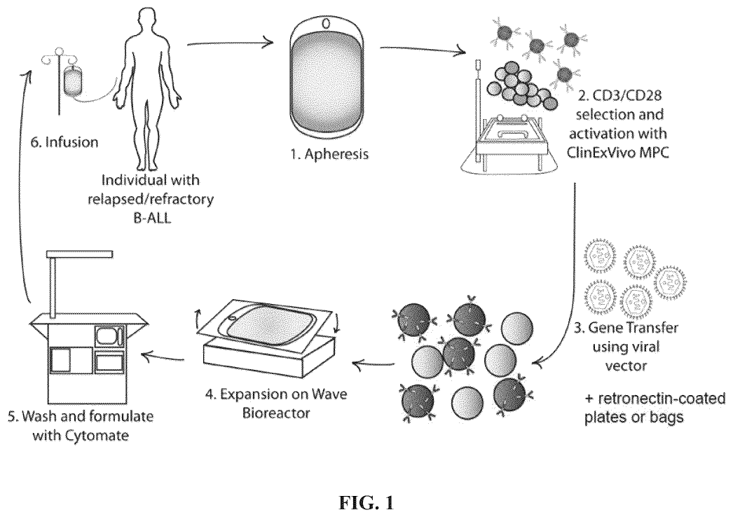

FIG. 2 illustrates critical reagents for prior art GMP CAR-T cell production.

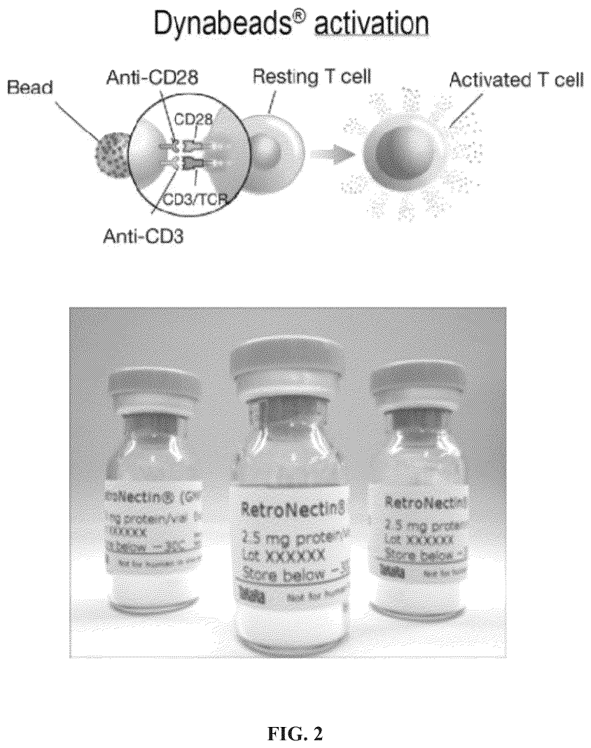

FIG. 3 illustrates how RetroNectin binds T cells and virus.

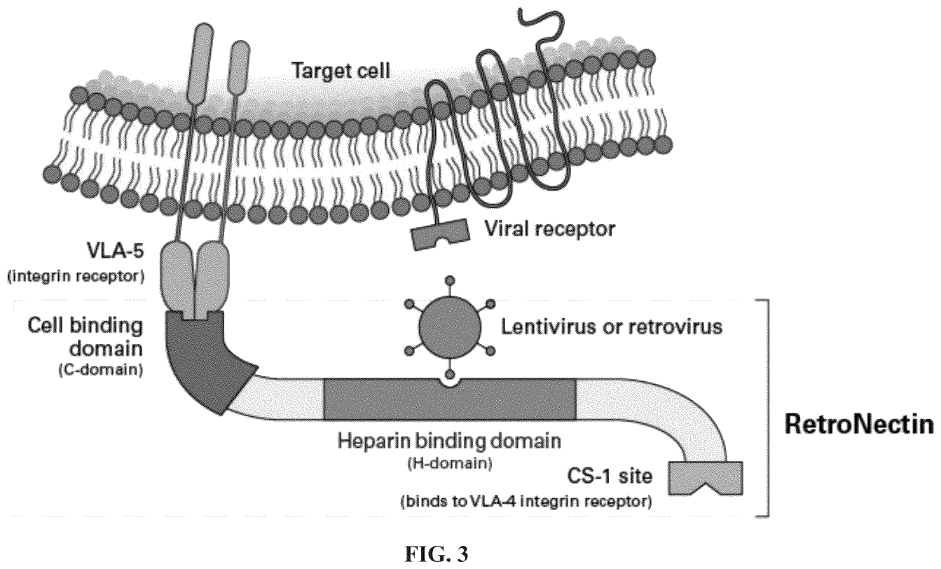

FIG. 4 illustrates an example synthetic antibody to engage CD3 or CD28 that contains a heparin binding domain.

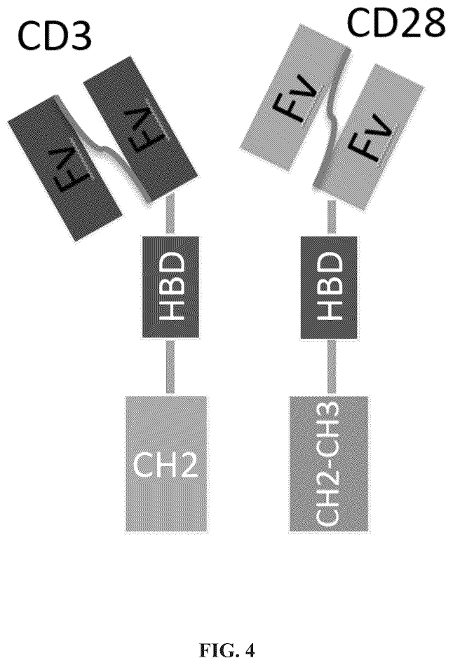

### DETAILED DESCRIPTION

Disclosed are fusion polypeptides comprising 1) variable domains of antibodies that specifically bind an immune cell antigen and 2) a heparin binding domain (HBD). The fusion polypeptides may comprise for example, at least one single chain antibody (scFv), at least one Fab fragment, at least one antibody, at least one Fv fragment, or any combination thereof. The scFv, Fab, or antibody encoded by the fusion polypeptide may be bivalent, trivalent, tetravalent, etc. Where a particular scFv is disclosed and said polypeptide are, it is understood that the multivalent antibodies can be multi-specific, e.g., bispecific, trispecific, tetraspecific, etc. The multivalent antibodies may be in any form, such as a diabody, triabody, tetrabody, etc.

Single chain antibodies may contain a heavy chain comprising one or more variable regions and/or a light chain comprising one or more variable regions. Single chain antibodies can be constructed using only antibody variable domains. A fairly efficient and relatively simple method is to make the linker sequence between the V_{H} and V_{L} domains so short that they cannot fold over and bind one another. Reduction of the linker length to 3-12 residues prevents the monomeric configuration of the scFv molecule and favors intermolecular VH-VL pairings with formation of a 60 kDa non-covalent scFv dimer “diabody”. The diabody format can also be used for generation of recombinant bi-specific antibodies, which are obtained by the noncovalent association of two single-chain fusion products, consisting of the VH domain from one antibody connected by a short linker to the VL domain of another antibody. Reducing the linker length still further below three residues can result in the formation of trimers (“triabody”, about 90 kDa) or tetramers (“tetrabody”, about 120 kDa). For a review of engineered antibodies, particularly single domain fragments, see Holliger and Hudson, 2005, Nature Biotechnology, 23:1126-1136. All of such engineered antibodies may be used in the fusion polypeptides provided herein.

Peptide linkers (-) suitable for production of scFv antibodies are described in Kumada Y, et al. Biochemical Engineering Journal. 2007 35 (2): 158-165; Albrecht H, et al. J Immunol Methods. 2006 310 (1-2): 100-16; Feng J, et al. J Immunol Methods. 2003 282 (1-2): 33-43; Griffiths A D, et al. Curr Opin Biotechnol. 1998 9 (1): 102-8; Huston J S, et al. Methods Enzymol. 1991 203:46-88; Bird R E, et al. Science. 1988 242 (4877): 423-6; Takkinen K, et al. Protein Eng. 1991 4 (7): 837-41; Smallshaw J E, et al. Protein Eng. 1999 12 (7): 623-30; Argos P. J Mol Biol. 1990 211 (4): 943-58; and Whitlow M, et al. Protein Eng. 1993 6 (8): 989-95, which are hereby incorporated by reference for the teachings of these linkers and methods of producing scFv antibodies against different targets using various linkers.

Tetravalent Tandab® may be prepared substantially as described in WO 1999057150 A3 or US20060233787, which are incorporated by reference for the teaching of methods of making Tandab® molecules.

The antigen recognition sites or entire variable regions of the engineered antibodies may be derived from one or more parental antibodies directed against any antigen of interest. The parental antibodies can include naturally occurring antibodies or antibody fragments, antibodies or antibody fragments adapted from naturally occurring antibodies, antibodies constructed de novo using sequences of antibodies or antibody fragments known to be specific for an antigen of interest. Sequences that may be derived from parental antibodies include heavy and/or light chain variable regions and/or CDRs, framework regions or other portions thereof.

The particular length of the peptide linker (-) used to join the scFv molecules to the heparin binding domain is important in determining half-life, immunogenicity, and activity of the overall construct. In some embodiments, the linker sequence (-) is 4, 5, 6, 7, 8, 9, 10, 11, 12, 13, 14, 15, 16, 17, 18, 19, 20, 21, 22, 23, 24, 25 or more amino acids in length. In some embodiments, the linker sequence (-) comprises GGGGS (SEQ ID NO:4). In some cases, the linker comprises 2, 3, 4, 5, or more GGGGS sequences. The linker is preferably long enough to not interfere with proper folding and association of the VH-VL chains but not so long as to cause added immunogenicity.

Candidate engineered antibodies for inclusion in the fusion polypeptides, or the fusion polypeptides themselves, may be screened for activity using a variety of known assays. For example, screening assays to determine binding specificity are well known and routinely practiced in the art. For a comprehensive discussion of such assays, see Harlow et al. (Eds.), ANTIBODIES: A LABORATORY MANUAL; Cold Spring Harbor Laboratory; Cold Spring Harbor, N.Y., 1988, Chapter 6.

In some embodiments, the bispecific antibody may be subjected to an alteration to render it less immunogenic when administered to a human. Such an alteration may comprise one or more of the techniques commonly known as chimerization, humanization, CDR-grafting, deimmunization and/or mutation of framework region amino acids to correspond to the closest human germline sequence (germlining). Bispecific antibodies which have been altered will therefore remain administrable for a longer period of time with reduced or no immune response-related side effects than corresponding bispecific antibodies which have not undergone any such alteration(s). One of ordinary skill in the art will understand how to determine whether, and to what degree an antibody must be altered in order to prevent it from eliciting an unwanted host immune response.

In some embodiments, the disclosed fusion polypeptide comprises a construct set forth in Table 1, 2, 3, or 4.

TABLE 1
CD3-targeting constructs

| | | |
|---|---|---|
| CD3 scFv | HBD | CH1 |
| CD3 scFv | HBD | CH2 |
| CD3 scFv | HBD | CH3 |
| CD3 scFv | HBD | CH1—CH2 |
| CD3 scFv | HBD | CH1—CH3 |
| CD3 scFv | HBD | CH2—CH1 |
| CD3 scFv | HBD | CH2—CH3 |
| CD3 scFv | HBD | CH3—CH1 |
| CD3 scFv | HBD | CH3—CH2 |
| CD3 scFv | HBD | CH1—CH2—CH3 |
| CD3 scFv | HBD | CH1—CH3—CH2 |
| CD3 scFv | HBD | CH2—CH1—CH3 |
| CD3 scFv | HBD | CH2—CH3—CH1 |
| CD3 scFv | HBD | CH3—CH1—CH2 |
| CD3 scFv | HBD | CH3—CH2—CH1 |
| CH1 | HBD | CD3 scFv |
| CH2 | HBD | CD3 scFv |
| CH3 | HBD | CD3 scFv |
| CH1—CH2 | HBD | CD3 scFv |
| CH1—CH3 | HBD | CD3 scFv |
| CH2—CH1 | HBD | CD3 scFv |
| CH2—CH3 | HBD | CD3 scFv |
| CH3—CH1 | HBD | CD3 scFv |
| CH3—CH2 | HBD | CD3 scFv |
| CH1—CH2—CH3 | HBD | CD3 scFv |
| CH1—CH3—CH2 | HBD | CD3 scFv |
| CH2—CH1—CH3 | HBD | CD3 scFv |
| CH2—CH3—CH1 | HBD | CD3 scFv |
| CH3—CH1—CH2 | HBD | CD3 scFv |
| CH3—CH2—CH1 | HBD | CD3 scFv |
| HBD | CD3 scFv | CH1 |
| HBD | CD3 scFv | CH2 |
| HBD | CD3 scFv | CH3 |
| HBD | CD3 scFv | CH1—CH2 |
| HBD | CD3 scFv | CH1—CH3 |
| HBD | CD3 scFv | CH2—CH1 |
| HBD | CD3 scFv | CH2—CH3 |
| HBD | CD3 scFv | CH3—CH1 |
| HBD | CD3 scFv | CH3—CH2 |
| HBD | CD3 scFv | CH1—CH2—CH3 |
| HBD | CD3 scFv | CH1—CH3—CH2 |
| HBD | CD3 scFv | CH2—CH1—CH3 |
| HBD | CD3 scFv | CH2—CH3—CH1 |
| HBD | CD3 scFv | CH3—CH1—CH2 |
| HBD | CD3 scFv | CH3—CH2—CH1 |

TABLE 2
CD28-targeting constructs:

| | | |
|---|---|---|
| CD28 scFv | HBD | CH1 |
| CD28 scFv | HBD | CH2 |
| CD28 scFv | HBD | CH3 |
| CD28 scFv | HBD | CH1—CH2 |
| CD28 scFv | HBD | CH1—CH3 |
| CD28 scFv | HBD | CH2—CH1 |
| CD28 scFv | HBD | CH2—CH3 |
| CD28 scFv | HBD | CH3—CH1 |
| CD28 scFv | HBD | CH3—CH2 |
| CD28 scFv | HBD | CH1—CH2—CH3 |
| CD28 scFv | HBD | CH1—CH3—CH2 |
| CD28 scFv | HBD | CH2—CH1—CH3 |
| CD28 scFv | HBD | CH2—CH3—CH1 |
| CD28 scFv | HBD | CH3—CH1—CH2 |
| CD28 scFv | HBD | CH3—CH2—CH1 |
| CH1 | HBD | CD28 scFv |
| CH2 | HBD | CD28 scFv |
| CH3 | HBD | CD28 scFv |
| CH1—CH2 | HBD | CD28 scFv |
| CH1—CH3 | HBD | CD28 scFv |
| CH2—CH1 | HBD | CD28 scFv |
| CH2—CH3 | HBD | CD28 scFv |
| CH3—CH1 | HBD | CD28 scFv |
| CH3—CH2 | HBD | CD28 scFv |
| CH1—CH2—CH3 | HBD | CD28 scFv |
| CH1—CH3—CH2 | HBD | CD28 scFv |
| CH2—CH1—CH3 | HBD | CD28 scFv |
| CH2—CH3—CH1 | HBD | CD28 scFv |
| CH3—CH1—CH2 | HBD | CD28 scFv |
| CH3—CH2—CH1 | HBD | CD28 scFv |
| HBD | CD28 scFv | CH1 |
| HBD | CD28 scFv | CH2 |
| HBD | CD28 scFv | CH3 |
| HBD | CD28 scFv | CH1—CH2 |
| HBD | CD28 scFv | CH1—CH3 |
| HBD | CD28 scFv | CH2—CH1 |
| HBD | CD28 scFv | CH2—CH3 |
| HBD | CD28 scFv | CH3—CH1 |
| HBD | CD28 scFv | CH3—CH2 |
| HBD | CD28 scFv | CH1—CH2—CH3 |
| HBD | CD28 scFv | CH1—CH3—CH2 |
| HBD | CD28 scFv | CH2—CH1—CH3 |
| HBD | CD28 scFv | CH2—CH3—CH1 |
| HBD | CD28 scFv | CH3—CH1—CH2 |
| HBD | CD28 scFv | CH3—CH2—CH1 |

TABLE 3
CD137/4-1BB-targeting constructs:

| | | |
|---|---|---|
| CD137 scFv | HBD | CH1 |
| CD137 scFv | HBD | CH2 |
| CD137 scFv | HBD | CH3 |
| CD137 scFv | HBD | CH1—CH2 |
| CD137 scFv | HBD | CH1—CH3 |
| CD137 scFv | HBD | CH2—CH1 |
| CD137 scFv | HBD | CH2—CH3 |
| CD137 scFv | HBD | CH3—CH1 |
| CD137 scFv | HBD | CH3—CH2 |
| CD137 scFv | HBD | CH1—CH2—CH3 |
| CD137 scFv | HBD | CH1—CH3—CH2 |
| CD137 scFv | HBD | CH2—CH1—CH3 |
| CD137 scFv | HBD | CH2—CH3—CH1 |
| CD137 scFv | HBD | CH3—CH1—CH2 |
| CD137 scFv | HBD | CH3—CH2—CH1 |
| CH1 | HBD | CD137 scFv |
| CH2 | HBD | CD137 scFv |
| CH3 | HBD | CD137 scFv |
| CH1—CH2 | HBD | CD137 scFv |
| CH1—CH3 | HBD | CD137 scFv |
| CH2—CH1 | HBD | CD137 scFv |
| CH2—CH3 | HBD | CD137 scFv |
| CH3—CH1 | HBD | CD137 scFv |
| CH3—CH2 | HBD | CD137 scFv |
| CH1—CH2—CH3 | HBD | CD137 scFv |
| CH1—CH3—CH2 | HBD | CD137 scFv |
| CH2—CH1—CH3 | HBD | CD137 scFv |
| CH2—CH3—CH1 | HBD | CD137 scFv |
| CH3—CH1—CH2 | HBD | CD137 scFv |
| CH3—CH2—CH1 | HBD | CD137 scFv |
| HBD | CD137 scFv | CH1 |
| HBD | CD137 scFv | CH2 |
| HBD | CD137 scFv | CH3 |
| HBD | CD137 scFv | CH1—CH2 |
| HBD | CD137 scFv | CH1—CH3 |
| HBD | CD137 scFv | CH2—CH1 |
| HBD | CD137 scFv | CH2—CH3 |
| HBD | CD137 scFv | CH3—CH1 |
| HBD | CD137 scFv | CH3—CH2 |
| HBD | CD137 scFv | CH1—CH2—CH3 |
| HBD | CD137 scFv | CH1—CH3—CH2 |
| HBD | CD137 scFv | CH2—CH1—CH3 |
| HBD | CD137 scFv | CH2—CH3—CH1 |
| HBD | CD137 scFv | CH3—CH1—CH2 |
| HBD | CD137 scFv | CH3—CH2—CH1 |

TABLE 4
CD3- and CD28- and CD137/4-1BB-targeting constructs:

| | | | | |
|---|---|---|---|---|
| CD3 scFv | CD28 scFv | CD137 scFv | HBD | CH1 |
| CD3 scFv | CD28 scFv | CD137 scFv | HBD | CH2 |
| CD3 scFv | CD28 scFv | CD137 scFv | HBD | CH3 |
| CD3 scFv | CD28 scFv | CD137 scFv | HBD | CH1—CH2 |
| CD3 scFv | CD28 scFv | CD137 scFv | HBD | CH1—CH3 |
| CD3 scFv | CD28 scFv | CD137 scFv | HBD | CH2—CH1 |
| CD3 scFv | CD28 scFv | CD137 scFv | HBD | CH2—CH3 |
| CD3 scFv | CD28 scFv | CD137 scFv | HBD | CH3—CH1 |
| CD3 scFv | CD28 scFv | CD137 scFv | HBD | CH3—CH2 |
| CD3 scFv | CD28 scFv | CD137 scFv | HBD | CH1—CH2—CH3 |
| CD3 scFv | CD28 scFv | CD137 scFv | HBD | CH1—CH3—CH2 |
| CD3 scFv | CD28 scFv | CD137 scFv | HBD | CH2—CH1—CH3 |
| CD3 scFv | CD28 scFv | CD137 scFv | HBD | CH2—CH3—CH1 |
| CD3 scFv | CD28 scFv | CD137 scFv | HBD | CH3—CH1—CH2 |
| CD3 scFv | CD28 scFv | CD137 scFv | HBD | CH3—CH2—CH1 |
| CD3 scFv | CD137 scFv | CD28 scFv | HBD | CH1 |
| CD3 scFv | CD137 scFv | CD28 scFv | HBD | CH2 |
| CD3 scFv | CD137 scFv | CD28 scFv | HBD | CH3 |
| CD3 scFv | CD137 scFv | CD28 scFv | HBD | CH1—CH2 |
| CD3 scFv | CD137 scFv | CD28 scFv | HBD | CH1—CH3 |
| CD3 scFv | CD137 scFv | CD28 scFv | HBD | CH2—CH1 |
| CD3 scFv | CD137 scFv | CD28 scFv | HBD | CH2—CH3 |
| CD3 scFv | CD137 scFv | CD28 scFv | HBD | CH3—CH1 |
| CD3 scFv | CD137 scFv | CD28 scFv | HBD | CH3—CH2 |
| CD3 scFv | CD137 scFv | CD28 scFv | HBD | CH1—CH2—CH3 |
| CD3 scFv | CD137 scFv | CD28 scFv | HBD | CH1—CH3—CH2 |
| CD3 scFv | CD137 scFv | CD28 scFv | HBD | CH2—CH1—CH3 |
| CD3 scFv | CD137 scFv | CD28 scFv | HBD | CH2—CH3—CH1 |
| CD3 scFv | CD137 scFv | CD28 scFv | HBD | CH3—CH1—CH2 |
| CD3 scFv | CD137 scFv | CD28 scFv | HBD | CH3—CH2—CH1 |
| CD28 scFv | CD3 scFv | CD137 scFv | HBD | CH1 |
| CD28 scFv | CD3 scFv | CD137 scFv | HBD | CH2 |
| CD28 scFv | CD3 scFv | CD137 scFv | HBD | CH3 |
| CD28 scFv | CD3 scFv | CD137 scFv | HBD | CH1—CH2 |
| CD28 scFv | CD3 scFv | CD137 scFv | HBD | CH1—CH3 |
| CD28 scFv | CD3 scFv | CD137 scFv | HBD | CH2—CH1 |
| CD28 scFv | CD3 scFv | CD137 scFv | HBD | CH2—CH3 |
| CD28 scFv | CD3 scFv | CD137 scFv | HBD | CH3—CH1 |
| CD28 scFv | CD3 scFv | CD137 scFv | HBD | CH3—CH2 |
| CD28 scFv | CD3 scFv | CD137 scFv | HBD | CH1—CH2—CH3 |
| CD28 scFv | CD3 scFv | CD137 scFv | HBD | CH1—CH3—CH2 |
| CD28 scFv | CD3 scFv | CD137 scFv | HBD | CH2—CH1—CH3 |
| CD28 scFv | CD3 scFv | CD137 scFv | HBD | CH2—CH3—CH1 |
| CD28 scFv | CD3 scFv | CD137 scFv | HBD | CH3—CH1—CH2 |
| CD28 scFv | CD3 scFv | CD137 scFv | HBD | CH3—CH2—CH1 |
| CD28 scFv | CD137 scFv | CD3 scFv | HBD | CH1 |
| CD28 scFv | CD137 scFv | CD3 scFv | HBD | CH2 |
| CD28 scFv | CD137 scFv | CD3 scFv | HBD | CH3 |
| CD28 scFv | CD137 scFv | CD3 scFv | HBD | CH1—CH2 |
| CD28 scFv | CD137 scFv | CD3 scFv | HBD | CH1—CH3 |
| CD28 scFv | CD137 scFv | CD3 scFv | HBD | CH2—CH1 |
| CD28 scFv | CD137 scFv | CD3 scFv | HBD | CH2—CH3 |
| CD28 scFv | CD137 scFv | CD3 scFv | HBD | CH3—CH1 |
| CD28 scFv | CD137 scFv | CD3 scFv | HBD | CH3—CH2 |
| CD28 scFv | CD137 scFv | CD3 scFv | HBD | CH1—CH2—CH3 |
| CD28 scFv | CD137 scFv | CD3 scFv | HBD | CH1—CH3—CH2 |
| CD28 scFv | CD137 scFv | CD3 scFv | HBD | CH2—CH1—CH3 |
| CD28 scFv | CD137 scFv | CD3 scFv | HBD | CH2—CH3—CH1 |
| CD28 scFv | CD137 scFv | CD3 scFv | HBD | CH3—CH1—CH2 |
| CD28 scFv | CD137 scFv | CD3 scFv | HBD | CH3—CH2—CH1 |
| CD137 scFv | CD3 scFv | CD28 scFv | HBD | CH1 |
| CD137 scFv | CD3 scFv | CD28 scFv | HBD | CH2 |
| CD137 scFv | CD3 scFv | CD28 scFv | HBD | CH3 |
| CD137 scFv | CD3 scFv | CD28 scFv | HBD | CH1—CH2 |
| CD137 scFv | CD3 scFv | CD28 scFv | HBD | CH1—CH3 |
| CD137 scFv | CD3 scFv | CD28 scFv | HBD | CH2—CH1 |
| CD137 scFv | CD3 scFv | CD28 scFv | HBD | CH2—CH3 |
| CD137 scFv | CD3 scFv | CD28 scFv | HBD | CH3—CH1 |
| CD137 scFv | CD3 scFv | CD28 scFv | HBD | CH3—CH2 |
| CD137 scFv | CD3 scFv | CD28 scFv | HBD | CH1—CH2—CH3 |
| CD137 scFv | CD3 scFv | CD28 scFv | HBD | CH1—CH3—CH2 |
| CD137 scFv | CD3 scFv | CD28 scFv | HBD | CH2—CH1—CH3 |
| CD137 scFv | CD3 scFv | CD28 scFv | HBD | CH2—CH3—CH1 |
| CD137 scFv | CD3 scFv | CD28 scFv | HBD | CH3—CH1—CH2 |
| CD137 scFv | CD3 scFv | CD28 scFv | HBD | CH3—CH2—CH1 |
| CD137 scFv | CD28 scFv | CD3 scFv | HBD | CH1 |
| CD137 scFv | CD28 scFv | CD3 scFv | HBD | CH2 |
| CD137 scFv | CD28 scFv | CD3 scFv | HBD | CH3 |
| CD137 scFv | CD28 scFv | CD3 scFv | HBD | CH1—CH2 |
| CD137 scFv | CD28 scFv | CD3 scFv | HBD | CH1—CH3 |
| CD137 scFv | CD28 scFv | CD3 scFv | HBD | CH2—CH1 |
| CD137 scFv | CD28 scFv | CD3 scFv | HBD | CH2—CH3 |
| CD137 scFv | CD28 scFv | CD3 scFv | HBD | CH3—CH1 |
| CD137 scFv | CD28 scFv | CD3 scFv | HBD | CH3—CH2 |
| CD137 scFv | CD28 scFv | CD3 scFv | HBD | CH1—CH2—CH3 |
| CD137 scFv | CD28 scFv | CD3 scFv | HBD | CH1—CH3—CH2 |
| CD137 scFv | CD28 scFv | CD3 scFv | HBD | CH2—CH1—CH3 |
| CD137 scFv | CD28 scFv | CD3 scFv | HBD | CH2—CH3—CH1 |
| CD137 scFv | CD28 scFv | CD3 scFv | HBD | CH3—CH1—CH2 |
| CD137 scFv | CD28 scFv | CD3 scFv | HBD | CH3—CH2—CH1 |
| CH1 | HBD | CD3 scFv | CD28 scFv | CD137 scFv |
| CH2 | HBD | CD3 scFv | CD28 scFv | CD137 scFv |
| CH3 | HBD | CD3 scFv | CD28 scFv | CD137 scFv |
| CH1—CH2 | HBD | CD3 scFv | CD28 scFv | CD137 scFv |
| CH1—CH3 | HBD | CD3 scFv | CD28 scFv | CD137 scFv |
| CH2—CH1 | HBD | CD3 scFv | CD28 scFv | CD137 scFv |
| CH2—CH3 | HBD | CD3 scFv | CD28 scFv | CD137 scFv |
| CH3—CH1 | HBD | CD3 scFv | CD28 scFv | CD137 scFv |
| CH3—CH2 | HBD | CD3 scFv | CD28 scFv | CD137 scFv |
| CH1—CH2—CH3 | HBD | CD3 scFv | CD28 scFv | CD137 scFv |
| CH1—CH3—CH2 | HBD | CD3 scFv | CD28 scFv | CD137 scFv |
| CH2—CH1—CH3 | HBD | CD3 scFv | CD28 scFv | CD137 scFv |
| CH2—CH3—CH1 | HBD | CD3 scFv | CD28 scFv | CD137 scFv |
| CH3—CH1—CH2 | HBD | CD3 scFv | CD28 scFv | CD137 scFv |
| CH3—CH2—CH1 | HBD | CD3 scFv | CD28 scFv | CD137 scFv |
| CH1 | HBD | CD3 scFv | CD137 scFv | CD28 scFv |
| CH2 | HBD | CD3 scFv | CD137 scFv | CD28 scFv |
| CH3 | HBD | CD3 scFv | CD137 scFv | CD28 scFv |
| CH1—CH2 | HBD | CD3 scFv | CD137 scFv | CD28 scFv |
| CH1—CH3 | HBD | CD3 scFv | CD137 scFv | CD28 scFv |
| CH2—CH1 | HBD | CD3 scFv | CD137 scFv | CD28 scFv |
| CH2—CH3 | HBD | CD3 scFv | CD137 scFv | CD28 scFv |
| CH3—CH1 | HBD | CD3 scFv | CD137 scFv | CD28 scFv |
| CH3—CH2 | HBD | CD3 scFv | CD137 scFv | CD28 scFv |
| CH1—CH2—CH3 | HBD | CD3 scFv | CD137 scFv | CD28 scFv |
| CH1—CH3—CH2 | HBD | CD3 scFv | CD137 scFv | CD28 scFv |
| CH2—CH1—CH3 | HBD | CD3 scFv | CD137 scFv | CD28 scFv |
| CH2—CH3—CH1 | HBD | CD3 scFv | CD137 scFv | CD28 scFv |
| CH3—CH1—CH2 | HBD | CD3 scFv | CD137 scFv | CD28 scFv |
| CH3—CH2—CH1 | HBD | CD3 scFv | CD137 scFv | CD28 scFv |
| CH1 | HBD | CD28 scFv | CD3 scFv | CD137 scFv |
| CH2 | HBD | CD28 scFv | CD3 scFv | CD137 scFv |
| CH3 | HBD | CD28 scFv | CD3 scFv | CD137 scFv |
| CH1—CH2 | HBD | CD28 scFv | CD3 scFv | CD137 scFv |
| CH1—CH3 | HBD | CD28 scFv | CD3 scFv | CD137 scFv |
| CH2—CH1 | HBD | CD28 scFv | CD3 scFv | CD137 scFv |
| CH2—CH3 | HBD | CD28 scFv | CD3 scFv | CD137 scFv |
| CH3—CH1 | HBD | CD28 scFv | CD3 scFv | CD137 scFv |
| CH3—CH2 | HBD | CD28 scFv | CD3 scFv | CD137 scFv |
| CH1—CH2—CH3 | HBD | CD28 scFv | CD3 scFv | CD137 scFv |
| CH1—CH3—CH2 | HBD | CD28 scFv | CD3 scFv | CD137 scFv |
| CH2—CH1—CH3 | HBD | CD28 scFv | CD3 scFv | CD137 scFv |
| CH2—CH3—CH1 | HBD | CD28 scFv | CD3 scFv | CD137 scFv |
| CH3—CH1—CH2 | HBD | CD28 scFv | CD3 scFv | CD137 scFv |
| CH3—CH2—CH1 | HBD | CD28 scFv | CD3 scFv | CD137 scFv |
| CH1 | HBD | CD28 scFv | CD137 scFv | CD3 scFv |
| CH2 | HBD | CD28 scFv | CD137 scFv | CD3 scFv |
| CH3 | HBD | CD28 scFv | CD137 scFv | CD3 scFv |
| CH1—CH2 | HBD | CD28 scFv | CD137 scFv | CD3 scFv |
| CH1—CH3 | HBD | CD28 scFv | CD137 scFv | CD3 scFv |
| CH2—CH1 | HBD | CD28 scFv | CD137 scFv | CD3 scFv |
| CH2—CH3 | HBD | CD28 scFv | CD137 scFv | CD3 scFv |
| CH3—CH1 | HBD | CD28 scFv | CD137 scFv | CD3 scFv |
| CH3—CH2 | HBD | CD28 scFv | CD137 scFv | CD3 scFv |
| CH1—CH2—CH3 | HBD | CD28 scFv | CD137 scFv | CD3 scFv |
| CH1—CH3—CH2 | HBD | CD28 scFv | CD137 scFv | CD3 scFv |
| CH2—CH1—CH3 | HBD | CD28 scFv | CD137 scFv | CD3 scFv |
| CH2—CH3—CH1 | HBD | CD28 scFv | CD137 scFv | CD3 scFv |
| CH3—CH1—CH2 | HBD | CD28 scFv | CD137 scFv | CD3 scFv |
| CH3—CH2—CH1 | HBD | CD28 scFv | CD137 scFv | CD3 scFv |
| CH1 | HBD | CD137 scFv | CD3 scFv | CD28 scFv |
| CH2 | HBD | CD137 scFv | CD3 scFv | CD28 scFv |
| CH3 | HBD | CD137 scFv | CD3 scFv | CD28 scFv |
| CH1—CH2 | HBD | CD137 scFv | CD3 scFv | CD28 scFv |
| CH1—CH3 | HBD | CD137 scFv | CD3 scFv | CD28 scFv |
| CH2—CH1 | HBD | CD137 scFv | CD3 scFv | CD28 scFv |
| CH2—CH3 | HBD | CD137 scFv | CD3 scFv | CD28 scFv |
| CH3—CH1 | HBD | CD137 scFv | CD3 scFv | CD28 scFv |
| CH3—CH2 | HBD | CD137 scFv | CD3 scFv | CD28 scFv |
| CH1—CH2—CH3 | HBD | CD137 scFv | CD3 scFv | CD28 scFv |
| CH1—CH3—CH2 | HBD | CD137 scFv | CD3 scFv | CD28 scFv |
| CH2—CH1—CH3 | HBD | CD137 scFv | CD3 scFv | CD28 scFv |
| CH2—CH3—CH1 | HBD | CD137 scFv | CD3 scFv | CD28 scFv |
| CH3—CH1—CH2 | HBD | CD137 scFv | CD3 scFv | CD28 scFv |
| CH3—CH2—CH1 | HBD | CD137 scFv | CD3 scFv | CD28 scFv |
| CH1 | HBD | CD137 scFv | CD28 scFv | CD3 scFv |
| CH2 | HBD | CD137 scFv | CD28 scFv | CD3 scFv |
| CH3 | HBD | CD137 scFv | CD28 scFv | CD3 scFv |
| CH1—CH2 | HBD | CD137 scFv | CD28 scFv | CD3 scFv |
| CH1—CH3 | HBD | CD137 scFv | CD28 scFv | CD3 scFv |
| CH2—CH1 | HBD | CD137 scFv | CD28 scFv | CD3 scFv |
| CH2—CH3 | HBD | CD137 scFv | CD28 scFv | CD3 scFv |
| CH3—CH1 | HBD | CD137 scFv | CD28 scFv | CD3 scFv |
| CH3—CH2 | HBD | CD137 scFv | CD28 scFv | CD3 scFv |
| CH1—CH2—CH3 | HBD | CD137 scFv | CD28 scFv | CD3 scFv |
| CH1—CH3—CH2 | HBD | CD137 scFv | CD28 scFv | CD3 scFv |
| CH2—CH1—CH3 | HBD | CD137 scFv | CD28 scFv | CD3 scFv |
| CH2—CH3—CH1 | HBD | CD137 scFv | CD28 scFv | CD3 scFv |
| CH3—CH1—CH2 | HBD | CD137 scFv | CD28 scFv | CD3 scFv |
| CH3—CH2—CH1 | HBD | CD137 scFv | CD28 scFv | CD3 scFv |
| HBD | CD3 scFv | CD28 scFv | CD137 scFv | CH1 |
| HBD | CD3 scFv | CD28 scFv | CD137 scFv | CH2 |
| HBD | CD3 scFv | CD28 scFv | CD137 scFv | CH3 |
| HBD | CD3 scFv | CD28 scFv | CD137 scFv | CH1—CH2 |
| HBD | CD3 scFv | CD28 scFv | CD137 scFv | CH1—CH3 |
| HBD | CD3 scFv | CD28 scFv | CD137 scFv | CH2—CH1 |
| HBD | CD3 scFv | CD28 scFv | CD137 scFv | CH2—CH3 |
| HBD | CD3 scFv | CD28 scFv | CD137 scFv | CH3—CH1 |
| HBD | CD3 scFv | CD28 scFv | CD137 scFv | CH3—CH2 |
| HBD | CD3 scFv | CD28 scFv | CD137 scFv | CH1—CH2—CH3 |
| HBD | CD3 scFv | CD28 scFv | CD137 scFv | CH1—CH3—CH2 |
| HBD | CD3 scFv | CD28 scFv | CD137 scFv | CH2—CH1—CH3 |
| HBD | CD3 scFv | CD28 scFv | CD137 scFv | CH2—CH3—CH1 |
| HBD | CD3 scFv | CD28 scFv | CD137 scFv | CH3—CH1—CH2 |
| HBD | CD3 scFv | CD28 scFv | CD137 scFv | CH3—CH2—CH1 |
| HBD | CD3 scFv | CD137 scFv | CD28 scFv | CH1 |
| HBD | CD3 scFv | CD137 scFv | CD28 scFv | CH2 |
| HBD | CD3 scFv | CD137 scFv | CD28 scFv | CH3 |
| HBD | CD3 scFv | CD137 scFv | CD28 scFv | CH1—CH2 |
| HBD | CD3 scFv | CD137 scFv | CD28 scFv | CH1—CH3 |
| HBD | CD3 scFv | CD137 scFv | CD28 scFv | CH2—CH1 |
| HBD | CD3 scFv | CD137 scFv | CD28 scFv | CH2—CH3 |
| HBD | CD3 scFv | CD137 scFv | CD28 scFv | CH3—CH1 |
| HBD | CD3 scFv | CD137 scFv | CD28 scFv | CH3—CH2 |
| HBD | CD3 scFv | CD137 scFv | CD28 scFv | CH1—CH2—CH3 |
| HBD | CD3 scFv | CD137 scFv | CD28 scFv | CH1—CH3—CH2 |
| HBD | CD3 scFv | CD137 scFv | CD28 scFv | CH2—CH1—CH3 |
| HBD | CD3 scFv | CD137 scFv | CD28 scFv | CH2—CH3—CH1 |
| HBD | CD3 scFv | CD137 scFv | CD28 scFv | CH3—CH1—CH2 |
| HBD | CD3 scFv | CD137 scFv | CD28 scFv | CH3—CH2—CH1 |
| HBD | CD28 scFv | CD3 scFv | CD137 scFv | CH1 |
| HBD | CD28 scFv | CD3 scFv | CD137 scFv | CH2 |
| HBD | CD28 scFv | CD3 scFv | CD137 scFv | CH3 |
| HBD | CD28 scFv | CD3 scFv | CD137 scFv | CH1—CH2 |
| HBD | CD28 scFv | CD3 scFv | CD137 scFv | CH1—CH3 |
| HBD | CD28 scFv | CD3 scFv | CD137 scFv | CH2—CH1 |
| HBD | CD28 scFv | CD3 scFv | CD137 scFv | CH2—CH3 |
| HBD | CD28 scFv | CD3 scFv | CD137 scFv | CH3—CH1 |
| HBD | CD28 scFv | CD3 scFv | CD137 scFv | CH3—CH2 |
| HBD | CD28 scFv | CD3 scFv | CD137 scFv | CH1—CH2—CH3 |
| HBD | CD28 scFv | CD3 scFv | CD137 scFv | CH1—CH3—CH2 |
| HBD | CD28 scFv | CD3 scFv | CD137 scFv | CH2—CH1—CH3 |
| HBD | CD28 scFv | CD3 scFv | CD137 scFv | CH2—CH3—CH1 |
| HBD | CD28 scFv | CD3 scFv | CD137 scFv | CH3—CH1—CH2 |
| HBD | CD28 scFv | CD3 scFv | CD137 scFv | CH3—CH2—CH1 |
| HBD | CD28 scFv | CD137 scFv | CD3 scFv | CH1 |
| HBD | CD28 scFv | CD137 scFv | CD3 scFv | CH2 |
| HBD | CD28 scFv | CD137 scFv | CD3 scFv | CH3 |
| HBD | CD28 scFv | CD137 scFv | CD3 scFv | CH1—CH2 |
| HBD | CD28 scFv | CD137 scFv | CD3 scFv | CH1—CH3 |
| HBD | CD28 scFv | CD137 scFv | CD3 scFv | CH2—CH1 |
| HBD | CD28 scFv | CD137 scFv | CD3 scFv | CH2—CH3 |
| HBD | CD28 scFv | CD137 scFv | CD3 scFv | CH3—CH1 |
| HBD | CD28 scFv | CD137 scFv | CD3 scFv | CH3—CH2 |
| HBD | CD28 scFv | CD137 scFv | CD3 scFv | CH1—CH2—CH3 |
| HBD | CD28 scFv | CD137 scFv | CD3 scFv | CH1—CH3—CH2 |
| HBD | CD28 scFv | CD137 scFv | CD3 scFv | CH2—CH1—CH3 |
| HBD | CD28 scFv | CD137 scFv | CD3 scFv | CH2—CH3—CH1 |
| HBD | CD28 scFv | CD137 scFv | CD3 scFv | CH3—CH1—CH2 |
| HBD | CD28 scFv | CD137 scFv | CD3 scFv | CH3—CH2—CH1 |
| HBD | CD137 scFv | CD3 scFv | CD28 scFv | CH1 |
| HBD | CD137 scFv | CD3 scFv | CD28 scFv | CH2 |
| HBD | CD137 scFv | CD3 scFv | CD28 scFv | CH3 |
| HBD | CD137 scFv | CD3 scFv | CD28 scFv | CH1—CH2 |
| HBD | CD137 scFv | CD3 scFv | CD28 scFv | CH1—CH3 |
| HBD | CD137 scFv | CD3 scFv | CD28 scFv | CH2—CH1 |
| HBD | CD137 scFv | CD3 scFv | CD28 scFv | CH2—CH3 |
| HBD | CD137 scFv | CD3 scFv | CD28 scFv | CH3—CH1 |
| HBD | CD137 scFv | CD3 scFv | CD28 scFv | CH3—CH2 |
| HBD | CD137 scFv | CD3 scFv | CD28 scFv | CH1—CH2—CH3 |
| HBD | CD137 scFv | CD3 scFv | CD28 scFv | CH1—CH3—CH2 |
| HBD | CD137 scFv | CD3 scFv | CD28 scFv | CH2—CH1—CH3 |
| HBD | CD137 scFv | CD3 scFv | CD28 scFv | CH2—CH3—CH1 |
| HBD | CD137 scFv | CD3 scFv | CD28 scFv | CH3—CH1—CH2 |
| HBD | CD137 scFv | CD3 scFv | CD28 scFv | CH3—CH2—CH1 |
| HBD | CD137 scFv | CD28 scFv | CD3 scFv | CH1 |
| HBD | CD137 scFv | CD28 scFv | CD3 scFv | CH2 |
| HBD | CD137 scFv | CD28 scFv | CD3 scFv | CH3 |
| HBD | CD137 scFv | CD28 scFv | CD3 scFv | CH1—CH2 |
| HBD | CD137 scFv | CD28 scFv | CD3 scFv | CH1—CH3 |
| HBD | CD137 scFv | CD28 scFv | CD3 scFv | CH2—CH1 |
| HBD | CD137 scFv | CD28 scFv | CD3 scFv | CH2—CH3 |
| HBD | CD137 scFv | CD28 scFv | CD3 scFv | CH3—CH1 |
| HBD | CD137 scFv | CD28 scFv | CD3 scFv | CH3—CH2 |
| HBD | CD137 scFv | CD28 scFv | CD3 scFv | CH1—CH2—CH3 |
| HBD | CD137 scFv | CD28 scFv | CD3 scFv | CH1—CH3—CH2 |
| HBD | CD137 scFv | CD28 scFv | CD3 scFv | CH2—CH1—CH3 |
| HBD | CD137 scFv | CD28 scFv | CD3 scFv | CH2—CH3—CH1 |
| HBD | CD137 scFv | CD28 scFv | CD3 scFv | CH3—CH1—CH2 |
| HBD | CD137 scFv | CD28 scFv | CD3 scFv | CH3—CH2—CH1 |

Also disclosed is a method for gene transfer of an immune effector cell with a viral particle that involves co-culturing the immune effector cells with the viral particle and a fusion polypeptide disclosed herein. The disclosed antibodies can also be used in automated systems that are being developed for CAR T cell production, such as the MACs Prodigy.

### Definitions

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “bispecific antibody” refers to an antibody having two different antigen-binding regions defined by different antibody sequences. This can be understood as different target binding but includes as well binding to different epitopes in one target.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “isolated polypeptide” refers to a polypeptide, which may be prepared from recombinant DNA or RNA, or be of synthetic origin, some combination thereof, or which may be a naturally-occurring polypeptide, which (1) is not associated with proteins with which it is normally associated in nature, (2) is isolated from the cell in which it normally occurs, (3) is essentially free of other proteins from the same cellular source, (4) is expressed by a cell from a different species, or (5) does not occur in nature.

The term “isolated nucleic acid” refers to a polynucleotide of genomic, cDNA, synthetic, or natural origin or some combination thereof, which (1) is not associated with the cell in which the “isolated nucleic acid” is found in nature, or (2) is operably linked to a polynucleotide to which it is not linked in nature.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The term “protein” (if single-chain), “polypeptide” and “peptide” are used interchangeably herein when referring to a gene product, e.g., as may be encoded by a coding sequence. When referring to “polypeptide” herein, a person of skill in the art will recognize that a protein can be used instead, unless the context clearly indicates otherwise. A “protein” may also refer to an association of one or more polypeptides. By “gene product” is meant a molecule that is produced as a result of transcription of a gene. Gene products include RNA molecules transcribed from a gene, as well as proteins translated from such transcripts.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

It is understood and herein contemplated that the fusion polypeptides disclosed herein are particularly useful in the treatment of cancer. Accordingly, disclosed herein are methods for treating cancer in a subject that involves administering to the subject a therapeutically effective amount of a disclosed pharmaceutical composition. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer. The disclosed fusion polypeptides can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed fusion polypeptides can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed fusion polypeptides can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

It is further contemplated herein that the disclosed fusion polypeptides can be used in combination with any known cancer therapeutic for the treatment of a cancer including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate).

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

A number of embodiments of the invention have been described. Nevertheless, it will be understood that various modifications may be made without departing from the spirit and scope of the invention. Accordingly, other embodiments are within the scope of the following claims.

A number of embodiments of the invention have been described. Nevertheless, it will be understood that various modifications may be made without departing from the spirit and scope of the invention. Accordingly, other embodiments are within the scope of the following claims.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Protocol for scFVHBD Transduction

1. Add 100 μl of PBS diluted anti-CD3 and or anti-CD28 scFv+HBD proteins to each well of round-bottom 6-well assay plate: 1 μg/ml (final concentration)
2. Tightly cover the plate with Parafilm and incubate at 37° C. for 2 hr and keep at 4 overnight
3. Isolate T cells and culture on scFv+HBD-coated plates overnight at 37° C.
4. Spinoculation #1 (Day 2)
5. Harvest purified T-cells and spin cells at 700 g for 5 min.
6. Dispense 1 mL of cells (2 to 3 million T cells) onto each well of a 6-well plate that has been pre-coated with scFv+HBD-coated
7. Add 1 mL of virus into each well
8. Spin plates at 2000 g for 1 hr @ RT
9. Incubation
10. Transduction and Spinoculation #2 (Day 3)
11. Remove 850 μL of cultured media from each well
12. Add 1 mL of virus to each well
13. Spin plates at 2000 g for 1 hr @ RT
14. Incubation
15. Add c-RPMI media (Day 4)
16. Day 5 cells are ready for use

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1.-19. (canceled)

20. A method for gene transfer of an immune effector cell, comprising:
contacting the immune effector cell with a viral vector and a fusion polypeptide to form a mixture, wherein the fusion polypeptide comprises a heparin binding domain that binds the viral vector and at least one antigen-binding region that binds an immune cell antigen;
culturing the mixture under conditions effective for transduction.

21. The method of claim 20, wherein the immune effector cell is a member of the human lymphoid lineage.

22. The method of claim 20, wherein the immune effector cell is a member of the human myeloid lineage.

23. The method of claim 20, wherein the immune cell antigen comprises a cell surface molecule that is expressed on human NK cells, T cells, monocytes, macrophages or granulocytes.

24. The method of claim 23, wherein the cell surface molecule comprises CD2, CD3, CD16, CD64, CD89, CD137 (4-1BB), NKp30, NKp44, NKp46, NKp80 (KLR-F1), NKG2C or NKG2D.

25. The method of claim 20, wherein the immune cell antigen is selected from the group consisting of CD3, NKG2D, CD2, CD28, CD25, and CD137.

26. The method of claim 20, wherein the immune cell antigen is CD64 or CD89.

27. The method of claim 20, wherein the at least one antigen-binding regions comprise the amino acid sequence SEQ ID NO:1.

28. The method of claim 20, wherein the at least one antigen-binding regions comprise the amino acid sequence SEQ ID NO:2.

29. The method of claim 20, wherein the heparin binding domain comprises the amino acid sequence SEQ ID NO:3.

30. The method of claim 20, wherein the fusion polypeptide comprises a single chain antibody (scFv).

31. The method of claim 20, wherein the fusion polypeptide comprises the following formula:
V_{L}I-V_{H}I-HBD-Fc,
V_{H}I-V_{L}I-HBD-Fc,
HBD-V_{L}I-V_{H}I-FC,
HBD-V_{H}I-V_{L}I-Fc,
Fc-V_{L}I-V_{H}I-HBD,
Fc-V_{H}I-V_{L}I-HBD,
Fc-HBD-V_{L}I-V_{H}I, or
Fc-HBD-V_{H}I-V_{L}I,
wherein “V_{L}I” is a light chain variable domain specific for the immune cell antigen;
wherein “V_{H}I” is a heavy chain variable domain specific for the immune cell antigen;
wherein “HBD” is the heparin binding domain;
wherein “-” consists of a peptide linker or a peptide bond;
wherein “-” consists of a peptide linker or a peptide bond; and
wherein “Fc” consist of an optional Fc domain of an immunoglobulin.

32. The method of claim 20, wherein the fusion polypeptide comprise a first antigen-binding region that specifically binds a first immune cell antigen and a second antigen-binding region that specifically bind a second immune cell antigen, wherein the first immune cell antigen and second immune cell antigen are different.

33. The method of claim 32, wherein the first immune cell antigen and second immune cell antigen are each independently selected from the group consisting of CD3, CD28, and CD137.

34. The method of claim 20, wherein the fusion polypeptide further comprises a first antigen-binding region that specifically binds CD3, a second antigen-binding region that specifically binds CD28, and a third antigen-binding region that specifically binds CD137.


SEQUENCE LISTING (4 sequences)

SEQ ID NO: 1 - protein, 268 aa, synthetic construct
MASPLTRFLSLNLLLLGESIILGSGEAQVQLQESGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTVTVSSGGGGSGGGGSGGGGSDIQLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKR

SEQ ID NO: 2 - protein, 271 aa, synthetic construct
MASPLTRFLSLNLLLLGESIILGSGEAQVQLQQSGTELVKPASSVKISCKASGYTSTSNYMHWIRQQPGNGLEWIGRIYPGNGNTKYNQKFDGKATPTADKSSSTAYMQLSRLTFEDSAVYFCASAPLDYGGHIMDAWGQGTTVTVSSGGGGSGGGGSGGGGSDIQLTQSPAFLSASLGETVSIECLGSEDIYGYLAWYQQKPGKSPQLLIYVANRLQDGVPSRFSGSGSGTQYSLKISGMQPEDEGDYYCLQGSKFPLTFGSGTKLEIKR

SEQ ID NO: 3 - protein, 271 aa, synthetic construct
AIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 4 - protein, 5 aa, synthetic construct
GGGGS